(19) United States
(12) Patent Application Publication
(10) Pub. No.: US 2013/0066197 A1
(43) Pub. Date: Mar. 14, 2013

(54) SYSTEM AND METHOD FOR BLOOD VESSEL STENOSIS VISUALIZATION AND NAVIGATION

(71) Applicants: Celine Pruvot, Buc (FR); DeAnn M. Haas, Pewaukee, WI (US); Amy Lynette Deubig, Dousman, WI (US); Guillermo Ruiz, Paris (FR)

(72) Inventors: Celine Pruvot, Buc (FR); DeAnn M. Haas, Pewaukee, WI (US); Amy Lynette Deubig, Dousman, WI (US); Guillermo Ruiz, Paris (FR)

(21) Appl. No.: 13/230,913

(22) Filed: Sep. 13, 2011

(51) Int. Cl. A61B 6/00

(52) U.S. Cl. 600/427

(57) ABSTRACT

A system and method for blood vessel stenosis visualization and navigation is disclosed. The CT system includes a rotatable gantry having an opening to receive a patient, an x-ray source positioned on the rotatable gantry to project x-rays toward a region-of-interest (ROI) of the patient that includes a plurality of blood vessels, an x-ray detector positioned on the rotatable gantry to receive x-rays emitted by the x-ray source and attenuated by the ROI, and a DAS operably connected to the x-ray detector. The CT system also includes a computer programmed to obtain CT image data for the ROI, reconstruct a 3D image of the plurality of blood vessels from the CT image data, automatically detect pathologies in the plurality of blood vessels, and identify the pathologies in the plurality of blood vessels on the 3D image, with a severity of the pathology being indicated on the 3D image.

### BACKGROUND OF THE INVENTION

Embodiments of the invention relate generally to diagnostic imaging and, more particularly, to a method and apparatus that provides for automatically identifying and diagnosing blood vessel pathologies.

Typically, in computed tomography (CT) imaging systems, an x-ray source emits a fan-shaped beam toward a subject. The beam, after being attenuated by the subject, impinges upon an array of radiation detectors. The intensity of the attenuated beam radiation received at the detector array is typically dependent upon the attenuation of the x-ray beam by the subject. Each detector element of the detector array produces a separate electrical signal indicative of the attenuated beam received by each detector element. The electrical signals are transmitted to a data processing system for analysis which ultimately produces an image.

In one specific application, CT imaging can be used to visualize localized narrowing of vessels, such as coronary arteries, cerebral arteries, or other vessels or arteries, also known as stenosis. The diagnosis of stenosis, and the related decision of whether or how to clinically intervene, is often based on the assessed extent of the narrowing, such as the percentage reduction in the cross sectional area compared with neighboring portions of the vessel. When employing current CT imaging methods, however, such stenosis diagnoses can require a great deal of time on the part of medical personnel. That is, existing methods for the analysis of CT image data are semi-automatic and require a radiologist to perform a series of procedures step by step. For example, the radiologist analyzes blood vessels one by one by visually inspecting their lumen and looking for pathologies. This is a tedious, error-prone, and time consuming process, with it not being uncommon for the formulation of a diagnosis to take between 20 to 35 minutes per patient. As most doctors are engaged predominantly in doing triage, they mainly try to eliminate risk and reject healthy patients, and thus such lengthy diagnosis times are highly undesirable, as they negatively affect patient throughput.

Thus, what is needed is a method and a system for automatically identifying and displaying blood vessel pathologies in an efficient manner, so as to minimize the diagnosis time. What is additionally needed is a method and a system for automatically quantifying a level of stenosis in blood vessels.

### BRIEF DESCRIPTION OF THE INVENTION

Embodiments of the invention are directed to a method and apparatus that provide for accurate blood vessel stenosis visualization and navigation.

In accordance with one aspect of the invention, a CT system includes a rotatable gantry having an opening to receive a patient to be scanned, an x-ray source positioned on the rotatable gantry and configured to project x-rays toward a region-of-interest of the patient that includes a plurality of blood vessels, an x-ray detector positioned on the rotatable gantry and positioned to receive x-rays emitted by the x-ray source and attenuated by the region-of-interest, and a data acquisition system (DAS) operably connected to the x-ray detector. The CT system also includes a computer programmed to obtain CT image data for the region-of-interest, reconstruct a 3D image of the plurality of blood vessels from the CT image data, automatically detect pathologies in the plurality of blood vessels, and identify the pathologies in the plurality of blood vessels on the 3D image, with a severity of the pathology being indicated on the 3D image.

In accordance with another aspect of the invention, a non-transitory computer readable storage medium is provided having stored thereon a computer program comprising instructions that, when executed by a computer, cause the computer to cause an x-ray source in a computed tomography (CT) system to emit x-rays toward a patient during a scanning procedure and obtain CT image data for a region-of-interest of the patient from the scan, with the region-of-interest comprising a cardiac region including an aorta and coronaries of the patient. The instructions also cause the computer to generate and display a 3D image of a coronary tree that includes the aorta and coronaries of the patient from the CT image data, detect a lumen surface of each of the aorta and coronaries, and distort the lumen surface of each of the aorta and coronaries to model an ideal lumen surface. The instructions further cause the computer to determine a stenosis condition of each of the aorta and coronaries based on a comparison of the respective detected lumen surface and ideal lumen surface and indicate the stenosis condition of each of the aorta and coronaries on the 3D image of the coronary tree.

In accordance with yet another aspect of the invention, a method of computed tomography (CT) imaging includes acquiring CT image data for a patient region-of-interest, the region-of-interest including a blood vessel structure therein, analyzing the CT image data to identify voxels corresponding to the blood vessel structure in the region-of-interest, and generating a 3D arterial tree image of the patient from the identified voxels. The method also includes detecting a lumen surface of each blood vessel in the arterial tree and distorting the lumen surface of each blood vessel to model an ideal lumen surface. The method further includes identifying stenosed regions in the blood vessels based on a comparison of the respective detected lumen surface and ideal lumen surface and indicating, on the 3D arterial tree image, a stenosis severity of each of the identified stenosed regions.

Various other features and advantages will be made apparent from the following detailed description and the drawings.

### BRIEF DESCRIPTION OF THE DRAWINGS

The drawings illustrate preferred embodiments presently contemplated for carrying out the invention.

In the drawings:

FIG. 1 is a pictorial view of a CT imaging system for use with embodiments of the invention.

FIG. 2 is a block schematic diagram of the system illustrated in FIG. 1.

FIG. 3 is a graphical representation of a 3D coronary tree generated by the CT imaging system of FIGS. 1 and 2.

FIG. 4 is a graphical representation of a user selected portion of the 3D coronary tree of FIG. 3.

FIG. 5 is a flowchart of a technique for CT imaging for stenosis identification and diagnosis according to an embodiment of the invention.

FIG. 6 is a cross-sectional view of a blood vessel illustrating the lumen surface and tracking centerline according to an embodiment of the invention.

### DETAILED DESCRIPTION

The operating environment of the invention is described with respect to a sixty-four-slice computed tomography (CT) system. However, it will be appreciated by those skilled in the art that the invention is equally applicable for use with single-slice or other multi-slice configurations. Moreover, the invention will be described with respect to the detection and conversion of x-rays. However, one skilled in the art will further appreciate that the invention is equally applicable for the detection and conversion of other high frequency electromagnetic energy. An implementation is employable with a “third generation” CT scanner and/or other CT systems.

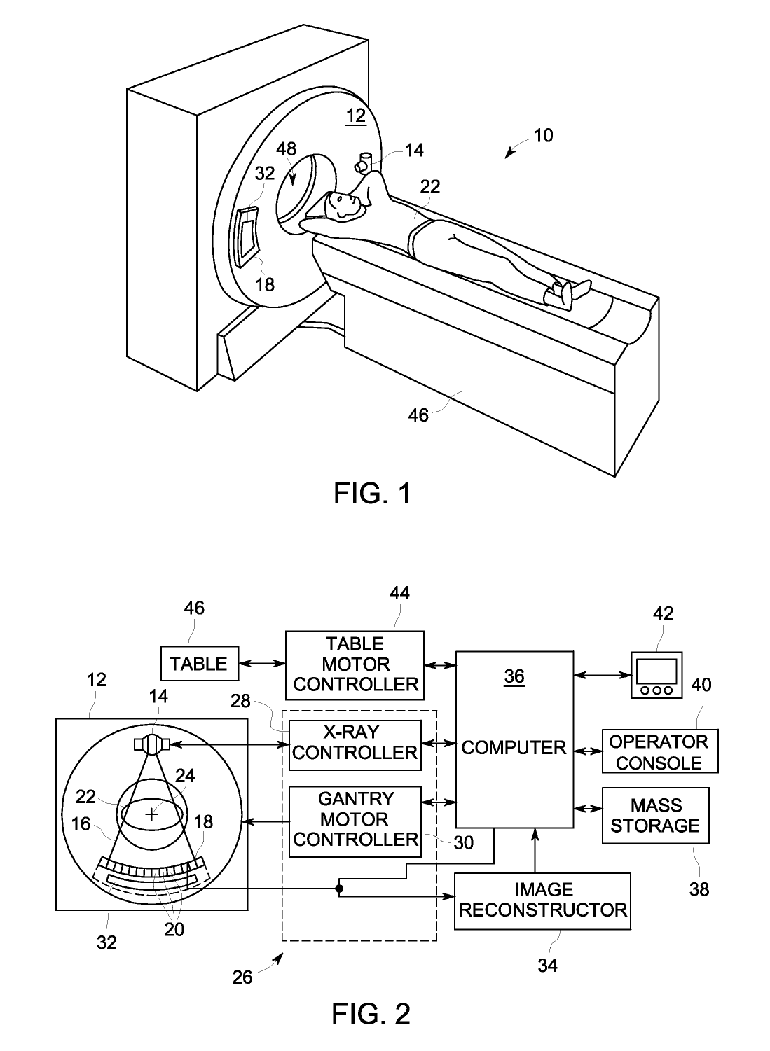

Referring to FIG. 1, a computed tomography (CT) imaging system 10 is shown as including a gantry 12 representative of a “third generation” CT scanner. According to an exemplary embodiment of the invention, CT system 10 is provided as a Gemstone Spectral Imaging (GSI) dual energy CT system from GE Healthcare. Gantry 12 has an x-ray source 14 that projects a beam of x-rays 16 toward a detector assembly or collimator 18 on the opposite side of the gantry 12. Referring now to FIG. 2, detector assembly 18 is formed by a plurality of detectors 20 and data acquisition systems (DAS) 32. The plurality of detectors 20 sense the projected x-rays that pass through a medical patient 22, with each detector 20 producing an analog electrical signal that represents the intensity of an impinging x-ray beam and hence the attenuated beam as it passes through the patient 22. The DAS 32 subsequently converts the analog electrical signal to digital signals, termed here as CT image data, for subsequent processing.

During a scan to acquire x-ray projection data, gantry 12 and the components mounted thereon rotate about a center of rotation 24. Rotation of gantry 12 and the operation of x-ray source 14 are governed by a control mechanism 26 of CT system 10. Control mechanism 26 includes an x-ray generator 28 that provides power and timing signals to an x-ray source 14 and a gantry motor controller 30 that controls the rotational speed and position of gantry 12. An image reconstructor 34 receives sampled and digitized x-ray data from DAS 32 and performs high speed reconstruction. The reconstructed image is applied as an input to a computer 36 which stores the image in a mass storage device 38.

Computer 36 also receives commands and scanning parameters from an operator via console 40 that has some form of operator interface, such as a keyboard, mouse, voice activated controller, or any other suitable input apparatus. An associated display 42 allows the operator to observe the reconstructed image and other data from computer 36. The operator supplied commands and parameters are used by computer 36 to provide control signals and information to DAS 32, x-ray generator 28 and gantry motor controller 30. In addition, computer 36 operates a table motor controller 44 which controls a motorized table 46 to position patient 22 and gantry 12. Particularly, table 46 moves patients 22 through a gantry opening 48 of FIG. 1 in whole or in part.

According to embodiments of the invention, computer 36 is further programmed to perform additional processing and analysis on the acquired CT image data. More specifically, computer 36 is programmed to automatically perform operations associated with analysis of blood vessel structures and identify/detect pathologies in the blood vessel structures, such as a partial stenosis of a coronary artery or vein from a plaque deposit. The computer 36 further determines a severity of the blood vessel stenosis and outputs an automated diagnosis regarding the severity, such that stenosis lesions in the coronary arties can be identified that might lead to an increased risk for myocardial infarction.

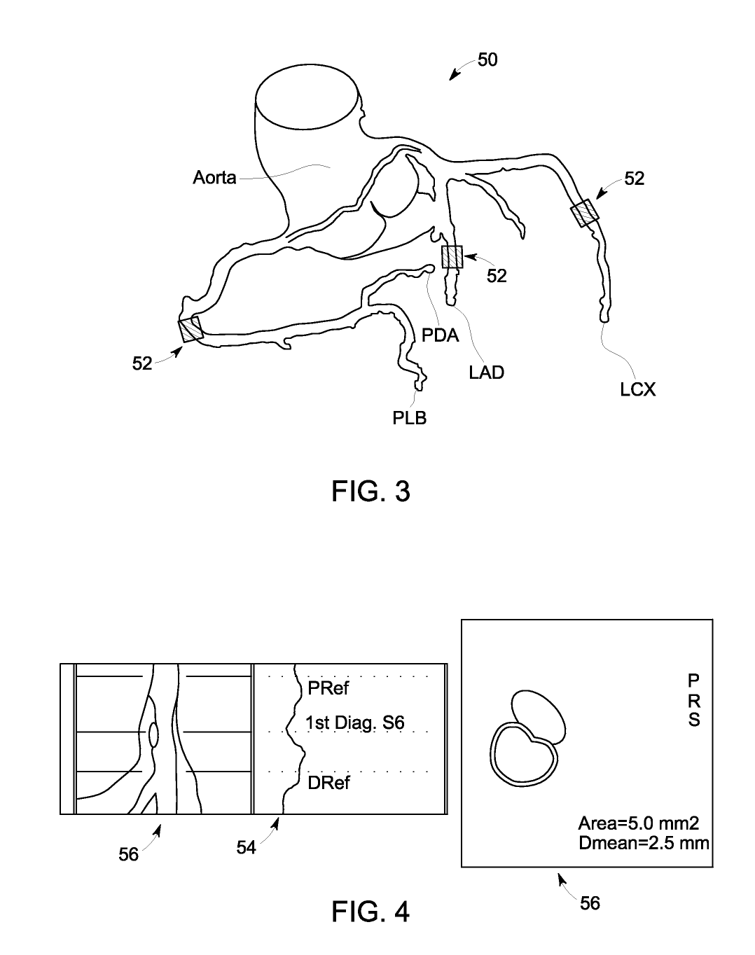

The computer 36 is further programmed to perform operations associated with presentation of the blood vessel analysis and identification results to a user. That is, computer 36 (along with image reconstructor 34) generates a 3D image of the coronary tree and causes the image to be displayed, such as on display 42. Referring to FIG. 3, an exemplary 3D image of the coronary tree 50 is provided. In displaying the 3D image of the coronary tree 50, computer 36 is further programmed to individually label the aorta and coronaries on the image. Thus, for example, the left anterior descending artery (LAD), left circumflex artery (LCx), posterior descending artery (PDA), posterior lateral branch (PLB), and aorta could be labeled on the coronary tree, along with other coronary arteries or veins.

As further shown in FIG. 3, the 3D image of the coronary tree includes thereon highlighted regions 52 that are representative of any stenosis lesions that have been identified/detected in the blood vessel structures and that are deemed to be significant. The highlighted regions 52 also include information regarding the severity of the identified/detected stenosis lesion. According to an exemplary embodiment of the invention, the highlighted regions 52 representative of stenosis lesions that have been identified/detected in the blood vessel structures are displayed on display in one of a number of colors according to a pre-determined color-coding scheme. For example, a green-yellow-red color scheme may be implemented for highlighting detected stenosis lesions, with stenosis lesions displayed in green having a “low” severity, stenosis lesions displayed in yellow having a “medium” severity, and stenosis lesions displayed in red having a “high” severity. Thresholds may be set for categorizing a stenosis lesion severity as low, medium, or high, with a percentage of blockage of a coronary artery or vein from a plaque deposit being used as a threshold setting.

According to one embodiment of the invention, upon a displaying of the 3D image of the coronary tree 50 on display 42, operator console 40 shown in FIG. 2 provides for a user selection of areas of interest 52 that are shown on the 3D image. That is, a user may, by way of operator console 40, click or select on a detected stenosis lesion 52 in the blood vessel structures that is highlighted on the 3D image of the coronary tree 50. Selection of a specific highlighted region 52 provides more detailed information on the selected stenosis region. As shown in FIG. 4, for example, user selection of a specific stenosis lesion results in the display of detailed data on the severity of the stenosis, such as specific measurement data 54 of the stenosis, as well as zoomed-in visualization(s) 56 of the stenosed region. The zoomed view(s) 56 can be rotated/translated by the user to optimize the view of the stenosed region. While operator console 40 is described above as providing for user interaction and selection of areas of interest that are shown on the 3D image of the coronary tree, it is recognized that other means of selection might also be provided. For example, display 42 may be in the form of a graphical user interface (GUI) or touch sensitive screen, by way of which a user could select a highlighted region on the 3D image of the coronary tree.

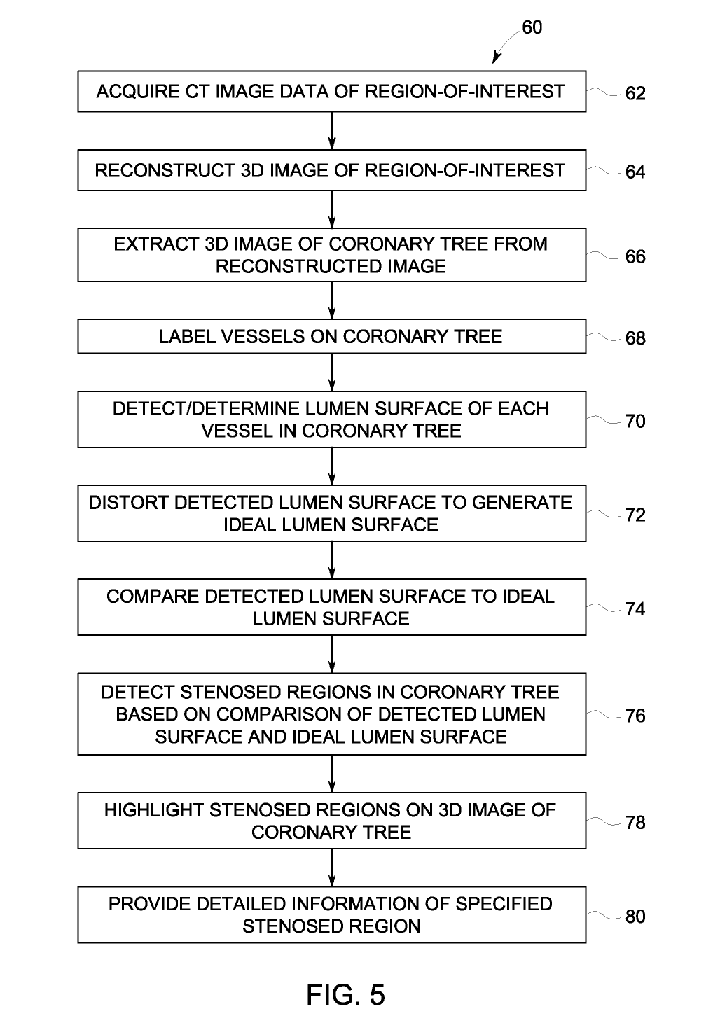

Referring now to FIG. 5, a computer implemented technique 60 for automatic diagnosis of a stenosis condition, such as would be implemented by computer 36 of CT system 10 of FIGS. 1 and 2, is shown according to an embodiment of the invention. According to embodiments of the invention, CT system 10 is operated according to technique 60 to obtain CT images of a region-of-interest of a patient that includes a blood vessel structure therein, such as a coronary artery or vein, that is partly stenosed (i.e., blocked) from a plaque deposit, so as allow for accurate blood vessel stenosis visualization and diagnosis to be made on the blood vessel.

At STEP 62, CT image data of a region-of-interest, such as a cardiac region, is acquired by the CT system. Image data can be acquired via one of any number of CT cardiac image acquisition techniques. For example, projection data from two or more heart cycles may be collected by CT system 10 for an angular range of at least 180 degrees plus a fan angle of the x-ray beam, with the timing of the data collection being chosen to be close to a desired phase of the cardiac cycle. Upon acquisition of the CT image data, one of several known image reconstruction methods is applied at STEP 64 to form a three-dimensional (3D) image of the region-of-interest. According to an exemplary embodiment, an image reconstruction technique that performs motion estimation and correction is employed, so as to minimize motion induced blurring and other motion artifacts in the image of the cardiac region.

It is recognized that the reconstructed 3D image not only shows the vascular tree, but also other structures such as bones. Therefore, further image processing is performed at STEP 66 to generate an isolated 3D image of the coronary tree/vascular tree from the CT image data according to one of several known techniques. For example, the aorta and coronaries can be extracted from the CT image data using one of a number of vessel segmentation techniques and algorithms. Examples of vessel segmentation algorithms and techniques that can be implemented include pattern recognition techniques, model-based approaches, tracking-based approaches, artificial intelligence-based approaches, and neural network-based approaches. Thus, techniques for extraction of the coronary tree may include steps such as thresholding followed by connected component analysis, or explicit vessel modeling to extract vessel contours, such as 3D surface representations for extracting convex and concave shapes on blood the vessel surfaces.

In addition to generating the coronary tree image, the aorta and coronaries on the image are labeled at STEP 68. According to one embodiment, upon segmenting of the previously acquired image(s) to generate the coronary tree, the generated coronary tree is compared with one or more coronary tree models to enable labeling of the coronary tree branches. The acquired coronary tree is labeled based on the labels corresponding to the modeled coronary tree having the closest similarity with the acquired coronary tree. The labeled coronary tree can then be displayed on the display 42 of the CT system 10 shown in FIG. 2.

Technique 60 continues by determining the lumen surface of each aorta and coronary at STEP 70. According to one embodiment, the lumen surface is determined by a method employing blood densities and shape constraints. That is, the lumen, for example, has a known density that can be measured based on the density of the blood flowing in the artery, and the density of the lumen surface is different than the density of calcifications and soft plaque that might be present in the blood vessel structure. As should be appreciated, the intensity values (i.e., CT numbers or Hounsfield numbers) of voxels that compose the 3D image are relative to tissue density. The lumen, wall of the vessel, calcification, and plaque, can thus be distinguished from one another based on their respective intensity values. Thus, separation of the lumen surface, calcifications, and soft plaque is accomplished by the use of intensity thresholds that define each structure.

According to one embodiment, determination of the lumen surface includes selecting a so-called seed pixel based on the distinct intensities, with the seed pixel being a pixel of the volumetric image data confidently known to be included in the vessel lumen. The seed pixel is used to perform a rough segmentation of the volumetric image data by the thresholding procedure set forth above. That is, the vessel interior is roughly designated by segmenting the vessel from the remaining tissue using a CT number threshold selected based on the distribution of the initial image pixels. Generally, the desired outcome of the segmenting operation is to distinguish regions of high intensity pixels from regions of relatively lower intensity pixels, or vice versa. The result of the segmenting operation is a roughly segmented lumen segment.

As part of this process, connectivity criteria are applied to the pixels meeting the threshold criterion. As will be appreciated by those of skill in the art, the connectivity criteria ensure that pixels that are not proximate to the roughly segmented lumen segment are not included in the output of the segmentation process. The threshold number is desirably selected by the user such that the vessel can be segmented from the adjoining material (e.g., organ tissues) while maintaining a continuous path along the length of the imaged portion of the vessel. More particularly, a point within the vessel is selected and threshold limits are selected to segment appropriately the vessel (such as an artery) from the remaining tissue (including structures in an organ supplied by the artery, for example) as described above. When the threshold is selected as a large fraction of the maximum pixel brightness in the lumen, regions outside the lumen are excluded by the rough segmentation operation. On the other hand, the threshold is also preferably selected to ensure that the rough lumen segment is continuous along the imaged portion length.

The rough segmentation provides an initial determination of the vessel lumen but is desirably designed to exclude marginal pixels. A more precise categorization of the pixels is then performed from the rough segmentation results by detecting contour voxels on the lumen surface (in a direction orthogonal to the local lumen centerline). That is, voxels may be considered as being on a contour voxel of the lumen surface if they are adjacent to at least one voxel that does not belong to the lumen surface. According to one embodiment, contour voxels are detected using morphological operations, which implement connectivity criteria and a so-called mask of the vessel lumen to detect the contour voxels. A still more accurate determination of the lumen surface may be achieved by performing of further processing. According to one embodiment, subvoxelic resolution of the lumen surface may be utilized, along with the application of contour and longitudinal smoothing, to achieve a more accurate lumen surface detection.

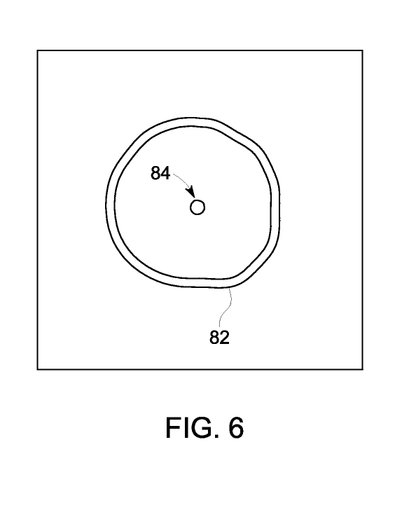

As part of the lumen surface detection, a tracking lumen centerline is also determined according to any suitable center line estimation method. While a rough estimation of the lumen centerline for each blood vessel in the coronary tree can be determined from the 3D image thereof, it is desirable to re-center the lumen centerline upon estimation of the lumen surface. Accurate identification of the vessel lumen surface provides for defining a true centerline of the lumen, negating the influence of any calcifications and plaque in the blood vessel that would inhibit tracking the centerline. A cross-sectional view of a blood vessel illustrating the lumen surface is provided in FIG. 6. According to the STEP 70 (FIG. 5) that is performed, the lumen surface 82 is detected, along with a tracking centerline 84.

Referring again to FIG. 5, technique 60 continues at STEP 72, where the detected lumen surface is distorted so as to generate an ideal or “healthy” lumen surface. A contouring or shape modification is performed on the detected lumen surface, such as by way of a force applied to tighten the lumen surface, to generate an ideal lumen surface that models what would be described as a healthy or stenosis-free lumen. Measurements on the ideal or healthy lumen surface are acquired and stored for use in a subsequent comparison with the detected lumen surface.

Thus, upon generation of an ideal lumen surface for each blood vessel in the coronary tree, technique 60 continues at STEP 74 with a comparison of the detected lumen surface to the ideal lumen surface for each vessel. According to one embodiment, the comparison provides for a determination of the degree of stenosis present in the vessel. That is, a measurement of the extent of stenosis can be determined based on the comparison of the detected lumen surface to the ideal lumen surface for each vessel, including radius/diameter measurements of the detected and ideal vessel lumen surfaces, as well as a determination of the percent blockage of the vessel caused by the stenosis.

Based on the comparison of the detected lumen surface to the ideal lumen surface for each vessel, stenosed regions that may be of concern are automatically identified and a stenosis severity for each identified stenosis region is determined at STEP 76. According to one embodiment of the invention, the identification and severity determinations are made by comparing the stenosis measurements/calculations (i.e., lumen radius/diameter measurements and/or percent blockage of the vessel) to one or more pre-determined threshold values. The severity of the stenosis for a vessel segment identified as a possible region of concern may be simply classified as “low,” “medium,” or “high,” according to one embodiment, although it is recognized that other more complex severity classifications may also be employed. Importantly, it is noted that each of STEPS 62-76 are performed automatically by computer 36, without input or calculations by the user.

In a next step of technique 60, the identified stenosed regions are highlighted on the displayed 3D image of the coronary tree at STEP 78. As indicated above, any stenosis regions that have been identified/detected in the blood vessel structures and that are deemed to be significant are highlighted to provide such data to a user/operator in an easy to assess fashion. According to one embodiment, the highlighted regions representative of the stenosed regions may be displayed in either a green, yellow, or red color based on the severity of the detected stenosis lesion. Stenosis lesions displayed in green have a “low” severity, stenosis lesions displayed in yellow have a “medium” severity, and stenosis lesions displayed in red have a “high” severity.

According to one embodiment of the invention, technique 60 continues at STEP 80, where detailed information on a user specified stenosed region is provided. That is, a user selection of one or more highlighted stenosed regions identified on the displayed 3D coronary tree is enabled, such as by way of a clicking on/selection of a such a region by way of the operator console 40 (FIG. 2). Selection of a specific highlighted region provides more detailed information on the selected stenosis region, such as the display of measurements of the stenosis and a zoomed-in visualization of the stenosed region.

A technical contribution for the disclosed method and apparatus is that is provides for a computer implemented method and apparatus for automatically identifying and displaying blood vessel pathologies in an efficient manner, so as to minimize the diagnosis time. The disclosed method and apparatus provides for a computer implemented method and apparatus for automatically quantifying and diagnosing a level of stenosis in blood vessels.

One skilled in the art will appreciate that embodiments of the invention may be interfaced to and controlled by a computer readable storage medium having stored thereon a computer program. The computer readable storage medium includes a plurality of components such as one or more of electronic components, hardware components, and/or computer software components. These components may include one or more computer readable storage media that generally stores instructions such as software, firmware and/or assembly language for performing one or more portions of one or more implementations or embodiments of a sequence. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or electronic memory. Other forms of non-transitory and/or tangible computer readable storage media not listed may be employed with embodiments of the invention.

A number of such components can be combined or divided in an implementation of a system. Further, such components may include a set and/or series of computer instructions written in or implemented with any of a number of programming languages, as will be appreciated by those skilled in the art.

Therefore, according to one embodiment of the invention, a CT system includes a rotatable gantry having an opening to receive a patient to be scanned, an x-ray source positioned on the rotatable gantry and configured to project x-rays toward a region-of-interest of the patient that includes a plurality of blood vessels, an x-ray detector positioned on the rotatable gantry and positioned to receive x-rays emitted by the x-ray source and attenuated by the region-of-interest, and a data acquisition system (DAS) operably connected to the x-ray detector. The CT system also includes a computer programmed to obtain CT image data for the region-of-interest, reconstruct a 3D image of the plurality of blood vessels from the CT image data, automatically detect pathologies in the plurality of blood vessels, and identify the pathologies in the plurality of blood vessels on the 3D image, with a severity of the pathology being indicated on the 3D image.

According to another embodiment of the invention, a non-transitory computer readable storage medium is provided having stored thereon a computer program comprising instructions that, when executed by a computer, cause the computer to cause an x-ray source in a computed tomography (CT) system to emit x-rays toward a patient during a scanning procedure and obtain CT image data for a region-of-interest of the patient from the scan, with the region-of-interest comprising a cardiac region including an aorta and coronaries of the patient. The instructions also cause the computer to generate and display a 3D image of a coronary tree that includes the aorta and coronaries of the patient from the CT image data, detect a lumen surface of each of the aorta and coronaries, and distort the lumen surface of each of the aorta and coronaries to model an ideal lumen surface. The instructions further cause the computer to determine a stenosis condition of each of the aorta and coronaries based on a comparison of the respective detected lumen surface and ideal lumen surface and indicate the stenosis condition of each of the aorta and coronaries on the 3D image of the coronary tree.

According to yet another embodiment of the invention, a method of computed tomography (CT) imaging includes acquiring CT image data for a patient region-of-interest, the region-of-interest including a blood vessel structure therein, analyzing the CT image data to identify voxels corresponding to the blood vessel structure in the region-of-interest, and generating a 3D arterial tree image of the patient from the identified voxels. The method also includes detecting a lumen surface of each blood vessel in the arterial tree and distorting the lumen surface of each blood vessel to model an ideal lumen surface. The method further includes identifying stenosed regions in the blood vessels based on a comparison of the respective detected lumen surface and ideal lumen surface and indicating, on the 3D arterial tree image, a stenosis severity of each of the identified stenosed regions.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A CT system comprising:
a rotatable gantry having an opening to receive a patient to be scanned;
an x-ray source positioned on the rotatable gantry and configured to project x-rays toward a region-of-interest of the patient that includes a plurality of blood vessels;
an x-ray detector positioned on the rotatable gantry and positioned to receive x-rays emitted by the x-ray source and attenuated by the region-of-interest;
a data acquisition system (DAS) operably connected to the x-ray detector; and
a computer programmed to:
obtain CT image data for the region-of-interest;
reconstruct a 3D image of the plurality of blood vessels from the CT image data;
automatically detect pathologies in the plurality of blood vessels; and
identify the pathologies in the plurality of blood vessels on the 3D image, with a severity of the pathology being indicated on the 3D image.

2. The CT system of claim 1 wherein the computer is further programmed to:
detect a lumen surface of each of the plurality of blood vessels;
distort the lumen surface of each of the plurality of blood vessels to model an ideal lumen surface;
compare the detected lumen surface of each of the plurality of blood vessels to the ideal lumen surfaces to identify plaque material in the plurality of blood vessels; and
automatically highlight, on the 3D image, stenosis lesions in the plurality of blood vessels based on the identified plaque material.

3. The CT system of claim 2 wherein the computer is further programmed to:
determine a severity of a stenosis lesion in each of the plurality of blood vessels based on the identified plaque material; and
highlight the stenosis lesions in the plurality of blood vessels in one of a plurality of pre-determined colors based on the determined severity of the stenosis lesions.

4. The CT system of claim 3 wherein the computer is further programmed to highlight the stenosis lesions in one of a green color, a yellow color, and a red color based on the determined severity of the stenosis lesions.

5. The CT system of claim 4 wherein the computer is further programmed to:
reconstruct a 3D image of a coronary tree from the CT image data; and
automatically identify and label aortas and coronaries in the 3D image of the coronary tree.

6. The CT system of claim 2 wherein the computer is further programmed to isolate the lumen surface of each of the plurality of blood vessels using one at least one of a blood density analysis and shape constraints.

7. The CT system of claim 6 wherein the computer is further programmed to:
determine a centerline of the lumen surface for each of the plurality of blood vessels;
detect contour voxels on the lumen surface for each of the plurality of blood vessels in a direction orthogonal to the centerline; and
apply contour and longitudinal smoothing to the contour voxels of the lumen surface for each of the plurality of blood vessels.

8. The CT system of claim 2 wherein the computer is further programmed to apply a force to each of the plurality of blood vessels to contour and modify a shape of the lumen surfaces, so as to generate the ideal lumen surfaces.

9. The CT system of claim 1 wherein the ideal lumen surface models a healthy blood vessel free of plaque.

10. The CT system of claim 1 further comprising an image display configured to display the reconstructed 3D image of the plurality of blood vessels.

11. The CT system of claim 10 further comprising a user input device configured to provide for user selection of the identified pathologies on the displayed 3D image, wherein user selection of an identified pathology causes pathology-related measurement data and zoomed visualizations related to the user selected pathology to be displayed on the image display.

12. A non-transitory computer readable storage medium having stored thereon a computer program comprising instructions, which, when executed by a computer, cause the computer to:
cause an x-ray source in a computed tomography (CT) system to emit x-rays toward a patient during a scanning procedure;
obtain CT image data for a region-of-interest of the patient from the scan, the region-of-interest comprising a cardiac region including an aorta and coronaries of the patient;
generate and display a 3D image of a coronary tree that includes the aorta and coronaries of the patient from the CT image data;
detect a lumen surface of each of the aorta and coronaries;
distort the lumen surface of each of the aorta and coronaries to model an ideal lumen surface;
determine a stenosis condition of each of the aorta and coronaries based on a comparison of the respective detected lumen surface and ideal lumen surface; and
indicate the stenosis condition of each of the aorta and coronaries on the 3D image of the coronary tree.

13. The non-transitory computer readable storage medium of claim 12 wherein the instructions further cause the computer to:
determine an extent of stenosis in each of the aorta and coronaries based on the comparison of respective detected lumen surfaces and ideal lumen surfaces;
compare the extent of stenosis to a plurality of pre-determined stenosis thresholds; and
highlight any portion of the aorta and coronaries in which the extent of stenosis exceeds one of the plurality of pre-determined stenosis thresholds, wherein the portion of the aorta and coronaries is highlighted in one of a plurality of pre-determined colors based on stenosis threshold that is exceeded.

14. The non-transitory computer readable storage medium of claim 12 wherein the instructions further cause the computer to automatically identify and label the aortas and coronaries in the 3D image of the coronary tree.

15. The non-transitory computer readable storage medium of claim 12 wherein the instructions further cause the computer to:
isolate the lumen surface of each of the aorta and the coronaries;
determine a centerline of the lumen surface for each of the aorta and the coronaries;
detect contour voxels on the lumen surface for each of the aorta and the coronaries in a direction orthogonal to the centerline; and
apply contour and longitudinal smoothing to the contour voxels of the lumen surface for each of the aorta and the coronaries.

16. The non-transitory computer readable storage medium of claim 12 wherein the instructions further cause the computer to apply a force to each of the aorta and the coronaries to contour and modify a shape of the lumen surfaces thereof, so as to generate the ideal lumen surfaces.

17. A method of computed tomography (CT) imaging comprising:
acquiring CT image data for a patient region-of-interest, the region-of-interest including a blood vessel structure therein;
analyzing the CT image data to identify voxels corresponding to the blood vessel structure in the region-of-interest;
generating a 3D arterial tree image of the patient from the identified voxels;
detecting a lumen surface of each blood vessel in the arterial tree;
distorting the lumen surface of each blood vessel to model an ideal lumen surface;
identifying stenosed regions in the blood vessels based on a comparison of the respective detected lumen surface and ideal lumen surface; and
indicating, on the 3D arterial tree image, a stenosis severity of each of the identified stenosed regions.

18. The method of claim 17 wherein detecting the lumen surface comprises:
isolating the lumen surface of each blood vessel using one at least one of a blood density analysis and shape constraints;
determining a centerline of the lumen surface for each of the blood vessels;
detecting contour voxels on the lumen surface for each of the blood vessels in a direction orthogonal to the centerline; and
applying contour and longitudinal smoothing to the lumen surface for each of the blood vessels.

19. The method of claim 17 wherein distorting the lumen surface comprises applying a force to each of the blood vessels to contour and modify a shape of the lumen surface, so as to generate the ideal lumen surfaces.

20. The method of claim 17 wherein indicating the stenosis severity comprises provided a color-coded indication of stenosis severity for each of the identified stenosed regions.

21. The method of claim 17 further comprising:
providing for user selection of the stenosed regions on the 3D image; and
displaying stenosis-related measurement data and zoomed visualizations for a user selected stenosed region.
